(19) United States
(12) Patent Application Publication
(10) Pub. No.: US 2025/0265314 A1
(43) Pub. Date: Aug. 21, 2025

(54) TARGET RECOGNITION METHODS BASED ON ELECTROENCEPHALOGRAM SIGNALS IN NATURAL READING ENVIRONMENT

(71) Applicant: TIANJIN UNIVERSITY, Tianjin (CN)

(72) Inventors: Yanru BAI, Tianjin (CN); Qi TANG, Tianjin (CN); Guangjian NI, Tianjin (CN); Dong MING, Tianjin (CN); Ran ZHAO, Tianjin (CN); Hongxing LIU, Tianjin (CN); Jinghan YANG, Tianjin (CN); Shihong JIANG, Tianjin (CN)

(73) Assignee: TIANJIN UNIVERSITY, Tianjin (CN)

(21) Appl. No.: 19/026,709

(22) Filed: Jan. 17, 2025

(30) Foreign Application Priority Data
Feb. 18, 2024 (CN) 202410181631.1

(51) Int. Cl. G06F 18/2413; G06F 3/01; G06F 18/10

(52) U.S. Cl. CPC G06F 18/2413; G06F 3/015; G06F 18/10; G06F 2218/04; G06F 2218/16

(57) ABSTRACT

Embodiments of the present disclosure provide a target recognition method based on an electroencephalogram (EEG) signal in a natural reading environment. The method includes steps 1-6. Step 1 includes determining a fuzzy semantic target recognition paradigm by selecting a stimulus material and designing an experimental Block. Step 2 includes performing an EEG experiment and acquiring the EEG signal according to the fuzzy semantic target recognition paradigm. Step 3 includes assessing quality of the acquired EEG signal and constructing an EEG database by combining a corresponding label. Step 4 includes obtaining a preprocessed EEG signal by performing preprocessing on the EEG signal in the EEG database. Step 5 includes performing feature extraction on the preprocessed EEG signal. Step 6 includes establishing an EEG classification model, and training and testing the established EEG classification model to recognize and classify a fuzzy semantic target in the natural reading environment.

### CROSS REFERENCE TO RELATED APPLICATIONS

The present disclosure claims priority to Chinese Patent Application No. 202410181631.1, filed on Feb. 18, 2024, the entire contents of which are incorporated herein by reference.

### TECHNICAL FIELD

The present disclosure relates to the field of cognitive science and artificial intelligence, and in particular, to target recognition methods based on electroencephalogram (EEG) signals in natural reading environment.

### BACKGROUND

Cognitive science is an advanced and cutting-edge discipline that studies human sensation, perception, mental states, brain thinking processes, and information processing. Research in this field is of great significance in uncovering the mysteries of the human brain. The classification of cognitive task patterns is widely used in the development of brain-machine interaction systems, the study of brain mechanisms, and the investigation of the pathogenesis of various brain diseases. Studies have shown that when subjects engage in a cognitive response related to a task, it involves complex cognitive processing stages, such as decision-making and working memory updating.

With the development of information technology, the importance of physiological signals has been discovered. Among them, EEG signals generated by the brain can reflect human brain activity, as well as information such as electrophysiological, and pathological. Specifically, when the brain is in an active state without specific external stimuli, the nervous system spontaneously generates rhythmic voltage changes, forming spontaneous EEG. Evoked EEG refers to the regular voltage changes in specific brain regions caused by external stimuli such as light, sound, electrical, or other types of task stimuli, which are known as event-related potentials (ERPs), also referred to as evoked potentials. Designing special experimental paradigms to conduct experiments, evoking EEG, and observing its variation process is an important method for brain cognition research. Analyzing EEG signals to study brain cognitive responses and exploring the relationship between semantic understanding and brain cognition has always been a key focus in EEG signal research.

Significant progress has been made in brain cognition research based on EEG signals. The main work includes studying the differences in working memory load and attention levels under different task conditions, the effects of emotions, cognition, and attention on ERPs, the decoding characteristics of specific brain regions on text content, the neural mechanisms of visual-spatial cognition under multitask conditions, and the assessment of neural damage in patients with mild cognitive impairment during task switching, etc. These studies involve both the impact of external stimuli on EEG and the analysis of brain neural mechanisms based on EEG signals.

In research on the brain's cognition of textual content, existing studies often adopt paradigms with clear objectives, usually focusing on specific texts. However, in natural reading environments, textual information may be semantically ambiguous, such as when the cognitive object refers to a category of things rather than a specific thing, which creates challenges for studying EEG signals in natural reading environments to analyze the brain's cognitive processes.

### SUMMARY

One or more embodiments of the present disclosure provide a target recognition method based on an electroencephalogram (EEG) signal in a natural reading environment. The method may comprise steps 1-6. Step 1 includes determining a fuzzy semantic target recognition paradigm by selecting a stimulus material and designing an experimental Block. Step 2 includes performing an EEG experiment and acquiring the EEG signal according to the fuzzy semantic target recognition paradigm. Step 3 includes assessing quality of the acquired EEG signal and constructing an EEG database by combining a corresponding label. Step 4 includes obtaining a preprocessed EEG signal by performing preprocessing on the EEG signal in the EEG database. Step 5 includes performing feature extraction on the preprocessed EEG signal. Step 6 includes establishing an EEG classification model, and training and testing an established EEG classification model to recognize and classify a fuzzy semantic target in the natural reading environment. In the step 6, the EEG classification model may include an EEGNet module, a CNN-LSTM module, a temporal feature module, a spatial feature module, and an integration module. The EEGNet module is configured to obtain a 1*30 feature vector using the preprocessed EEG signal obtained in step 4.4 as an input. The CNN-LSTM module is configured to obtain a 1*30 feature vector using a time-frequency feature set obtained in step 5.2 as an input and further extract a feature. The temporal feature module is configured to obtain a normalized 1*30 feature vector using a sample entropy feature vector calculated in step 5.3 as an input and normalize the sample entropy feature vector. The spatial feature module is configured to obtain a normalized 1*30 spatial feature vector using a spatial feature vector obtained in step 5.4 as an input and normalize the spatial feature vector. The integration module is configured to integrate the 1*30 feature vector obtained by the EEGNet module, the 1*30 feature vector obtained by the CNN-LSTM module, the normalized 1*30 feature vector obtained by the temporal feature module, and the normalized 1*30 spatial feature vector obtained by the spatial feature module into a whole integrated feature, and input the whole integrated feature into a fully connected layer; learn and fuse the integrated feature using the fully connected layer, and finally obtain a binary classification output after processing by a Softmax activation function layer. The EEGNet module may include a Batchnorm layer, two Modules A, a Dropout layer, the Fully connected layer, and a Flatten layer in turn. Each of the two Modules A includes the BatchNorm layer, the Dropout layer, a convolutional layer, a GlobalMaxpool layer, the Fully connected layer, a Relu layer, the Fully connected layer, a Sigmoid layer, and a Maxpool layer in turn. The CNN-LSTM module may include the BatchNorm layer, three Modules B, an LSTM layer, the Fully connected layer, and the Flatten layer. Each of the three Modules B includes the convolutional layer, the Relu layer, and the Maxpool layer in turn. The integration module includes the Fully connected layer, the Softmax layer, and binary classification.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, where:

FIG. 1 is a flowchart illustrating a process of an exemplary target recognition method based on an EEG signal in a natural reading environment according to some embodiments of the present disclosure;

FIG. 2 is a schematic diagram illustrating an exemplary stimulus material according to some embodiments of the present disclosure;

FIG. 3 is a schematic diagram illustrating an exemplary experimental process according to some embodiments of the present disclosure;

FIG. 4 is a schematic diagram illustrating an exemplary structure of an EEG classification model according to some embodiments of the present disclosure;

FIG. 5 is a schematic diagram illustrating an exemplary Module A in an EEG classification model according to some embodiments of the present disclosure; and

FIG. 6 is a schematic diagram illustrating an exemplary Module B in an EEG classification model according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure.

It should be understood that the terms “system”, “device”, “unit” and/or “module” as used herein are a way to distinguish between different components, elements, parts, sections, or assemblies at different levels. However, the words may be replaced by other expressions if other words accomplish the same purpose.

As shown in the present disclosure and the claims, unless the context clearly suggests an exception, the words “a”, “an”, “one” and/or “the” do not refer specifically to the singular but may also include the plural. Generally, the terms “including” and “comprising” suggest only the inclusion of clearly identified steps and elements that do not constitute an exclusive list, and the method or apparatus may also include other steps or elements.

It should be appreciated that the preceding or following operations are not necessarily performed in an exact sequence. Instead, steps can be processed in reverse order or simultaneously. Also, it is possible to add other operations to these processes or remove a step or steps from them.

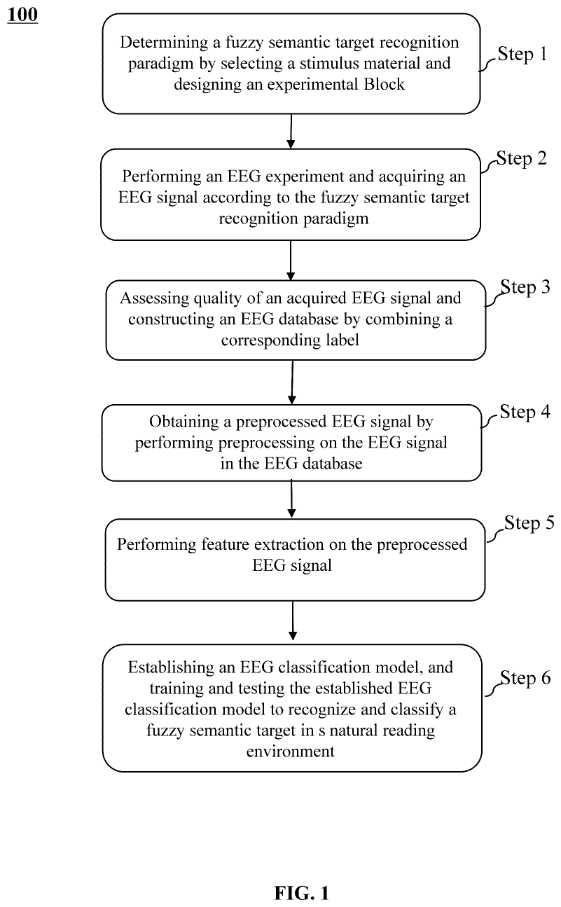

FIG. 1 is a flowchart illustrating a process of an exemplary target recognition method based on an EEG signal in a natural reading environment according to some embodiments of the present disclosure.

As shown in FIG. 1, a process 100 may include the following steps. In some embodiments, the process 100 may be executed by a processor.

The processor may process data and/or information related to the target recognition method based on the EEG signal in the natural reading environment. The processor may execute program instructions based on such data, information, and/or processing results to execute one or more of the functions described in the present disclosure. In some embodiments, the processor may include one or more sub-processing devices (e.g., a single-core processing device, a multi-core processing device, etc.). By way of example only, the processor may include a central processing unit (CPU), an application-specific integrated circuit (ASIC), etc., or any combination of the above.

In step 1: determining a fuzzy semantic target recognition paradigm by selecting a stimulus material and designing an experimental Block.

The stimulus material may be a material used to elicit an EEG signal.

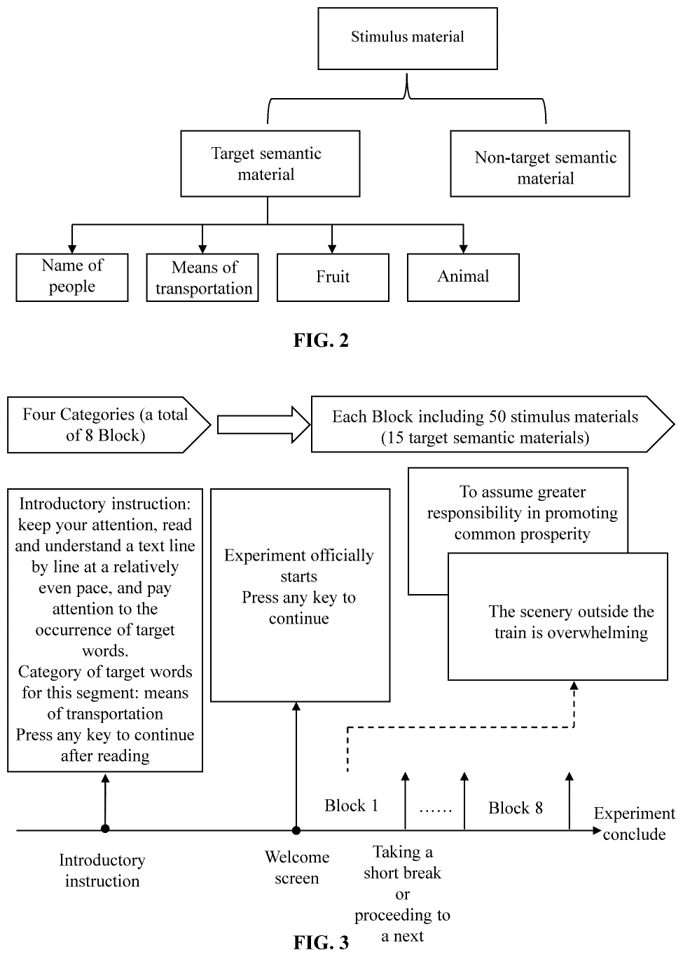

FIG. 2 is a schematic diagram illustrating an exemplary stimulus material according to some embodiments of the present disclosure.

In some embodiments, in the fuzzy semantic target recognition paradigm in the step 1, the stimulus material is a text of 15 to 20 words in length and is presented in a form of a news headline or a short sentence. As shown in FIG. 2, the stimulus material includes two categories: a target semantic material (target words) and a non-target semantic material (non-target words). To realize some coverage, the target semantic material (target words) includes four categories including a name of people, means of transportation, an animal, and a fruit. Each category is embodied as multiple target words, e.g., the category of “a name of people” includes two target words: “Zhang San” and “Li Si”. An experimental material is mainly sourced from online resources such as Baidu Baike, Renwu magazine, and WeChat public articles, and is homologous to a target work scenario. The specific content is summarized in Table 1. In order to form a contrast with the target words, the non-target words are selected from a more neutral news report, mainly from the media's reports related to the 20th National Congress of the Communist Party of China. A neutral material is chosen mainly to avoid interfering with an EEG feature of a subject due to its individual preferences.

TABLE 1
Categories of experimental materials and specific content thereof

| Cetegory | Specific Content | Content |
|---|---|---|
| Name of people | Zhang San, Li Si | 50*2 |
| Means of transportation | Train, Airplane, Ship, Bicyle, | 50*2 |
| | Bullet train | |
| Anminal | Parrot, Hedgehog, Crane, | 50*2 |
| | Steed, Kitten | |
| Fruit | Watermelon, Pineapple, | 50*2 |
| | Orange, Strawberyy, Grape | |

The target semantic material is a stimulus material used to elicit a cognitive reflection from the subject on the target words. The non-target semantic material is a stimulus material that avoids the individual preferences of the subject.

In some embodiments of the present disclosure, using the target semantic material and the non-target semantic material as the stimulus material is conducive to improving the validity and reliability of an EEG experiment.

In some embodiments, the processor may randomly select the stimulus material from a network resource.

The experimental Block is an experiment block formed by dividing the experimental material. An EEG experiment consists of a plurality of blocks, each block includes a plurality of trials, and the plurality of trials included in each block are run under a same condition in a preset order to minimize interference of random variability on the experiment.

The trial is a complete course of a single experimental manipulation or stimulus, including presentation of a stimulus material, response of a subject, and a collected EEG signal (same as EEG data). The subject refers to an experimenter who is subjected to the EEG experiment.

The fuzzy semantic target recognition paradigm may be a paradigm used to study how well the subject recognizes fuzzy semantics.

In some embodiments, the fuzzy semantic target recognition paradigm includes a total of 8 Blocks. In a single Block, a ratio of a target semantic material trial to a non-target semantic material trial may be 3:7, and an interval may exist between each trial.

In some embodiments, as shown in FIG. 3, stimulus materials under each target semantic category are randomly composed into 2 Blocks, resulting in a total of 8 Blocks designed. A single Block contains 50 stimulus materials, including 15 target word trials and 35 non-target word trials (a ratio of 3:7). Each Block may complete 50 trials, and there exists a 0.2 s interval between each trial, i.e., for each subject, 50 pieces of EEG data, each lasting 2.2 seconds (2 seconds for stimulus material presentation and 0.2 seconds for an interval), may be collected. In a single Block, the stimulus material appears pseudo-randomized, and it is guaranteed that a same category of target semantic material does not appear consecutively. In the entire Block of the experiment, all occurrences of stimulus material are not repeated.

In some embodiments of the present disclosure, a target semantic material trial and a non-target semantic material trial in a single Block are proportionally distributed. There exists an interval between each trial, which is conducive to better controlling a condition of the EEG experiment, ensuring the reliability and validity of the EEG experiment. At the same time, it can be ensured that each trial is more independent by setting the interval, improving the accuracy of the EEG signal.

In step 2: performing the EEG experiment and acquiring the EEG signal according to the fuzzy semantic target recognition paradigm.

In some embodiments, in the step 2, the EEG experiment includes 2*count of semantic categories*50 trials. Each trial presents a stimulus material, and a total of 2*count of semantic categories*50 stimulus materials are presented, wherein the stimulus material may be divided into the target semantic material and the non-target semantic material, e.g., the target semantic material accounts for 39% and the non-target semantic material accounts for 70%.

The whole EEG experiment includes 400 trials (50×8), totaling 400 stimulus materials, of which 120 are target semantic materials and 280 are non-target semantic materials.

After a short break, repeating step 2.2 and step 2.3, a stimulus material in a next Block is presented until all Blocks have been traversed.

FIG. 3 is a schematic diagram illustrating an exemplary experiment process according to some embodiments of the present disclosure.

As shown in FIG. 3, a subject begins an EEG experiment by first reading an introductory instruction. The instruction provides an overview of the experiment, including a requirement of the experiment and a category (e.g., means of transportation) of target words for a current segment. The subject then enters a welcome screen, marking the official start of the experiment. Next, the subject progresses to an experimental Block 1, where stimulus materials (e.g., a target semantic material such as “the scenery outside the train is overwhelming” and a non-target semantic material such as “to assume greater responsibility in promoting common prosperity,” etc.) in the Block 1 are presented. After the stimulus materials in the Block 1 have been presented, the subject may take a short break before proceeding to a next Block. After all Blocks have been traversed, the experiment concludes.

In some embodiments, the step 2 may include step 2.1 to step 2.3.

In step 2.1: wearing a wireless EEG acquisition device for the subject and informing the subject of a target semantic category.

The wireless EEG acquisition device may include a wireless electrode cap, a wireless EEG machine, or the like.

The target semantic category includes four categories of the target semantic material: name-related, transportation-related, animal-related, and fruit-related.

In step 2.2: randomly presenting a stimulus material in a single Block until all materials in the single Block have been traversed, and connecting a preceding material and a following material using a blank frame.

In step 2.3: recording an EEG signal and a corresponding label when presenting each stimulus material and an EEG signal and a corresponding label during a period of blank frame following each stimulus material.

For example, a presentation time of each stimulus material may be 2 s, and a time of the blank frame may be 0.2 s, the EEG signal may be a 30-lead signal, and the corresponding label may be whether a target word appears in the stimulus material. The corresponding label may be represented by 0 and 1, with 0 indicating that the target word does not appear in the stimulus material and 1 indicating that the target word appears in the stimulus material. The label may be marked manually.

In some embodiments, after a short break, repeating the step 2.2 and the step 2.3, the stimulus material in the next Block is represented until all Blocks have been traversed. A length of the break may be set by default or by a processor or by a technician depending on a situation of the subject.

In some embodiments of the present disclosure, by informing the subject of the target semantic category, traversing all materials within all experimental Blocks, and recording the EEG signal and the corresponding label when presenting each stimulus material and the EEG signal and the corresponding label during a period of blank frame following each stimulus material, which is conducive to ensuring that subject has a clear expectation for the experiment, controls a cognitive process of the subject, improves the analyzability of EEG signal data, and renders a more reliable experimental result.

In the step 3: assessing quality of the acquired EEG signal and constructing an EEG database by combining a corresponding label.

In some embodiments, the step 3 may include step 3.1 to step 3.5.

In step 3.1: calculating a power spectrum of each lead signal.

Lead refers to an electrode configuration manner, and different leads may record EEG signals in different regions of the brain. The lead signal refers to an EEG signal captured and recorded through the lead, i.e., the lead signal is a manifestation of the EEG signal.

The power spectrum of the lead signal refers to a plot of a power distribution of the lead signal in a frequency domain. In a power spectrum plot, an X-axis represents a signal frequency, and a Y-axis represents a power density corresponding to a frequency.

In some embodiments, the processor may calculate the power spectrum of each lead signal using a frequency-domain analysis manner. The frequency-domain analysis manner may include Fourier transform, or the like.

In step 3.2: labeling a lead signal with a value greater than twice a standard deviation of an average power spectral energy as a bad lead signal and supplementing using neighborhood interpolation.

The bad lead signal refers to a lead signal with a low-quality after being assessed.

In some embodiments, the processor may calculate a power spectrum energy of each lead signal based on the power spectral plot, calculate an average and standard deviation of power spectrum energies of a plurality of lead signals, and label the lead signal with a value greater than twice the standard deviation of the average power spectral energy as the bad lead signal and remove the bad lead signal, and supplement remaining lead signals using neighborhood interpolation, i.e., data of a bad lead signal is replaced by an average of neighboring lead signals.

In step 3.3: calculating, in a single trial, a median of a variance of lead signals, and a median of a difference between each lead signal and an average of the lead signals.

In step 3.4: labeling a trial in which either of the two medians is greater than twice a standard deviation and rejecting EEG data corresponding to the trial.

In step 3.5: corresponding retained EEG data with a label corresponding to the EEG data and constructing an EEG database.

An EEG signal (30-lead signals) during a presentation time (2 s) of each stimulus material and a period (0.2 s) of blank frame following the stimulus material may be one sample. Whether the target word appears in the stimulus material may be a sample label, with 0 indicating the target word does not appear and 1 indicating the target word appears.

In some embodiments of the present disclosure, by screening in terms of both the lead signal and the trial, removing data of the bad lead signal and rejecting EEG data corresponding to a portion of trials, and constructing the EEG database based on the screened EEG data and labels corresponding to the screened EEG data are conducive to providing high-quality data resources for the EEG database, thereby improving the recognition effect.

In some embodiments, assessing the quality of the acquired EEG signal in the step 3 may further include assessing the quality of the EEG signal based on a user-type feature of the subject, an acquisition time feature of the EEG signal, and the EEG signal.

The user-type feature may include gender, age, education level, or the like.

The acquisition time feature may be a feature related to an acquisition time of the EEG signal. For example, the acquisition time feature may be categorized as morning, afternoon, evening, or alternatively, within 4 hours, between 4 and 10 hours, and more than 10 hours since the subject's last break. Under different acquisition time features, the states of the subject are different, which has different effects on the acquisition of the EEG signal.

The quality of a signal may be a parameter that assesses how good or bad the quality of the EEG signal is. The quality of a signal may be expressed by a numerical value, and the larger the value, the better the quality of the EEG signal.

In some embodiments, the processor may construct a plurality of first reference vectors based on historical user-type features, historical acquisition time features, and historical EEG signals corresponding to a plurality of historical subjects in a plurality of historical EEG experiments; construct a first target vector based on a user-type feature, an acquisition time feature, and an EEG signal of a current subject; determine a first reference vector that has a greatest vectorial similarity with the first target vector, compare a historical EEG signal included in the first reference vector with an EEG signal of the historical EEG signal after a historical actual preprocessing to determine a signal difference (represented by a difference in data amount) between the historical EEG signal before and after the preprocessing, calculate the reciprocal of a product of the signal difference and a preprocessing intensity, and designate the reciprocal as the quality of a corresponding signal. The preprocessing intensity is positively correlated with a count of filtrations in the preprocessing.

In some embodiments, the processor may also assess the quality of the EEG signal based on the user-type feature, the acquisition time feature, and the EEG signal using a quality assessment model.

The quality assessment model may be a model used to assess the quality of an EEG signal. In some embodiments, the quality assessment model may be a machine learning model, e.g., a Convolutional Neural Network (CNN). An input of the quality assessment model may include the user-type feature, the acquisition time feature, and the EEG signal, and an output of the quality assessment model may be the quality of the EEG signal.

In some embodiments, the quality assessment model may be obtained by training based on a plurality of sets of first training samples with a first training label.

In some embodiments, a first training sample may be composed of a sample user-type feature, a sample acquisition time feature, and a sample EEG signal. The first training sample may be obtained based on historical data. For example, a first reference vector may serve as a first training sample. The first training label may be historical signal quality corresponding to the first training sample, which may be represented by the reciprocal of a product of a historical signal difference and a historical preprocessing intensity.

In some embodiments, the first training sample is input into an initial quality assessment model, a first loss function is constructed based on the signal quality output from the initial quality assessment model and the first training label, and the initial quality assessment model is updated based on the first loss function, and when a first preset condition is satisfied, the training of the initial quality assessment model is completed, then a trained quality assessment model is obtained. The first preset condition may be that the first loss function converges, a count of iterations reaches a threshold, or the like.

In some embodiments, the input of the quality assessment model may also include a stimulus material corresponding to an EEG signal.

In some embodiments, the first training sample further includes a sample stimulus material. The sample stimulus material may be obtained based on historical data. In some embodiments, the processor may train the quality assessment model based on the first training sample that includes the sample stimulus material.

In some embodiments of the present disclosure, using the stimulus material as the input of the quality assessment model can be effective in targeting the assessment of the quality of the EEG signal in a natural reading environment, and thus improve the accuracy of the assessment result of the quality assessment model.

In some embodiments, the step 2 further includes employing different ambient light intensity features and material carrier features for different experimental Blocks.

An ambient light intensity feature refers to a light feature of a surrounding environment in which the EEG experiment is performed, and the ambient light intensity feature may include a light brightness, a light color system (warm, cool, etc.), and so on.

A material carrier feature refers to a presentation carrier of the stimulus material. For example, if the stimulus material is a text, the material carrier feature may be a paper text or an electronic text, etc.

In some embodiments, the input of the quality assessment model may also include the ambient light intensity feature and the material carrier feature.

In some embodiments, the first training sample further includes a sample ambient light intensity feature and a sample material carrier feature corresponding to the sample EEG signal. The processor may train the quality assessment model based on the first training sample including the sample ambient light intensity feature and the sample material carrier feature.

In some embodiments of the present disclosure, considering the effects of different environments on the subject can be effective in targeting the assessment of the quality of the EEG signal in the natural reading environment, and thus improve the assessment effect of the quality assessment model.

In some embodiments of the present disclosure, the quality of a signal can be quickly and accurately determined using the quality assessment model, which is conducive to reducing computational resources occupied by obtaining a preprocessed EEG signal, thus reducing a time cost.

In some embodiments of the present disclosure, assessing the quality of the EEG signal based on the user-type feature, the acquisition time feature, and the EEG signal is conducive to reducing a random error, by fully considering individual differences in EEG signals among different subjects and personal features of the subjects, improving the accuracy of assessing the quality of the EEG signal.

In some embodiments, the quality of a signal is also configured to determine whether to reject the EEG signal. If the quality of a signal is lower than a quality threshold, an EEG signal corresponding to the signal may be rejected. The quality threshold may be predetermined by a technician based on experience.

In some embodiments, for a subject whose count of rejections exceeds a count threshold, the processor may re-conduct an experiment on the subject in accordance with a sampling parameter. The sampling parameter may include a subject and an acquisition time feature.

The count of rejections refers to a total count of EEG signals rejection times corresponding to a plurality of trials in a single EEG experiment of a subject. The larger the count of rejections, the more trials (EEG data corresponding to the trials) were rejected in that EEG experiment. The count threshold may be pre-set manually.

In some embodiments, the sampling parameter includes a sampling combination of the subject and an acquisition period.

In some embodiments, the processor may obtain a count of rejections of each subject in a respective EEG experiment. For a subject whose count of rejections exceeds the count threshold, the processor may re-obtain EEG data by re-conducting the EEG experiment on the subject based on an acquisition period feature.

In some embodiments, the sampling parameter further includes an ambient light intensity feature and a material carrier feature.

In some embodiments, the processor may also generate a plurality of candidate sampling parameters, determine a rejection probability of each of the plurality of candidate sampling parameters using a parameter determination model, and determine the sampling parameter based on the rejection probability.

In some embodiments, for each subject that needs to be re-take the EEG experiment, the processor may randomly combine ambient light intensity features, material carrier features, and acquisition period features corresponding to a plurality rejected EEG signals to determine a plurality of sets of candidate sampling parameters for each subject.

In some embodiments, the processor may also randomly combine ambient light intensity features, material carrier features, and acquisition period features corresponding to a plurality of rejected EEG signals of a plurality of subjects, and randomly match with a plurality of subjects that need to re-take the EEG experiment to determine a plurality of candidate sampling parameters corresponding to the plurality of subjects.

The rejection probability of a candidate sampling parameter refers to a probability that EEG data is rejected when the EEG experiment is conducted according to the candidate sampling parameter. The rejection probability may be a Boolean value in a range of 0 to 1, wherein 1 indicates being rejected and 0 indicates being not rejected.

The parameter determination model may be a model that determines the sampling parameter. The parameter determination model may be a machine learning model. For example, the parameter determination model may be a CNN, etc.

In some embodiments, an input of the parameter determination model may include the candidate sampling parameter, the stimulus material, an acquisition device parameter, and an output of the parameter determination model may be the rejection probability of the candidate sampling parameter.

In some embodiments, the parameter determination model may be obtained by training based on a plurality of sets of second training samples with a second training label.

In some embodiments, a second training sample may be composed of a sample sampling parameter, a sample stimulus material, and a sample acquisition device parameter. The second training sample may be obtained based on historical data. The second training label may be whether EEG data corresponding to the second training sample is rejected. The processor may determine whether a subsequently acquired historical EEG signal corresponding to the second training sample is rejected in accordance with step 3.3 and step 3.4, and the second training label is manually labeled.

The training of the parameter determination model may refer to the above training process of the quality assessment model, which will not be repeated here.

In some embodiments, the processor may randomly select one of one or more non-rejected candidate sampling parameters as the sampling parameter.

It should be noted in the embodiments of the present disclosure, the target semantic material may also be referred to as the target word, the non-target semantic material may also be referred to as the non-target word, the target semantic material trial may also be referred to as a target word trial, the non-target semantic material trial may also be referred to as a non-target word trial, the EEG data may also be referred to as EEG signal data or an EEG signal, and the lead signal may also be referred to as a lead EEG signal.

In some embodiments of the present disclosure, considering the ambient light intensity feature and the material carrier feature is conducive to simulating the natural reading environment and improving the quality of data in the EEG database.

In some embodiments of the present disclosure, performing resampling based on a count of times the EEG signals are rejected is conducive to fully considering different fluctuations of EEG signals of different subjects during different acquisition periods, thus determining a reasonable optimal combination, thereby obtaining an optimal EEG database.

In step 4: obtaining a preprocessed EEG signal by performing preprocessing on the EEG signal in the EEG database.

In some embodiments, the preprocessing in the step 4 includes filtering, downsampling, and denoising.

In some embodiments, a specific frequency of the EEG signal may be selected through filtering. For example, an unwanted frequency in the EEG signal is removed, and an interested frequency band of the EEG signal is retained. Filtering may be accomplished by, for example, a filter.

Downsampling refers to a process of reducing a sampling rate of the EEG signal to reduce an amount of data. Downsampling may be achieved through a downsampling function, etc.

Denoising refers to the reduction of noise interference in the EEG signal. Sources of noise may include eye movements, muscle movements, and so on. Denoising may be achieved by wavelet transform, independent component analysis (ICA), etc.

In some embodiments of the present disclosure, filtering, downsampling, and denoising the EEG data is conducive to suppressing the environmental interference, reducing the amount of data and a processing time, and improving a signal-to-noise ratio of the EEG signal, thereby improving the accuracy of feature extraction and analysis.

In some embodiments, the step 4 may include step 4.1 to step 4.4.

In step 4.1: obtaining a re-referenced EEG signal by re-referencing the EEG data using an average of all lead signals as a reference datum.

The average of all lead signals refers to an average of a plurality of lead signals in a same trial.

In step 4.2: obtaining a filtered EEG signal by removing, from the re-referenced EEG signal, noise below 0.5 Hz and above 80 Hz and power-line interference at 50 Hz using a band-pass filter and a notch filter.

In step 4.3: obtaining a downsampled EEG signal by downsampling the filtered EEG signal according to a sampling theorem.

In step 4.4: decomposing the downsampled EEG signal into a plurality of independent components using the ICA, calculating a frequency feature of each component; and removing bioelectrical artifacts and residual noise.

ICA refers to the ICA algorithm. For example, maximum likelihood estimation (MLE), information maximization, or the like.

The bioelectrical artifacts may be electrical activities that are not EEG signals produced by biological disturbances.

In some embodiments, the processor may utilize the ICA to decompose the downsampled EEG signal into a plurality of components that are independent of each other, e.g., a component that represents myoelectricity, a component that represents ocular electricity, etc., and set a discrimination threshold to remove each independent component in accordance with a probability, so as to remove the bioelectric artifacts and the residual noise.

In some embodiments of the present disclosure, the EEG signal is re-referenced based on the average of all lead signals, and then denoised using the band-pass filter and the notch filter; and downsampled and decomposed using the ICA to remove the bioelectrical artifacts and the residual noise, which is beneficial to reduce the periodic noise and improve the accuracy of recognizing and removing the bioelectric artifacts, thereby improving the quality of the EEG signal.

In step 5: performing feature extraction on the preprocessed EEG signal.

In some embodiments, the step 5 may include step 5.1 to step 5.4.

In step 5.1: obtaining a signal time-frequency plot of each lead by performing time-frequency analysis on the preprocessed EEG signal using continuous wavelet transform (CWT).

The signal time-frequency plot is a graph that shows the properties of the EEG signal in time and frequency. A horizontal coordinate of the signal time-frequency plot may denote time and a vertical coordinate of the signal time-frequency plot may denote frequency.

In step 5.2: obtaining a time-frequency feature set of an EEG signal of each lead by extracting an image feature of the signal time-frequency plot using a convolutional neural network.

The time-frequency feature set is a set of image features of the signal time-frequency plot.

In step 5.3: obtaining a sample entropy feature vector of each trial by calculating a sample entropy feature of a preprocessed signal of each lead in the trial.

The sample entropy feature refers to an eigenvalue of a sample entropy of a preprocessed EEG signal. The greater the sample entropy, the greater the regularity of brain activity. In some embodiments, the processor may extract the sample entropy feature through a sample entropy calculation formula.

The sample entropy feature vector may be a vector constructed from sample entropy features of a plurality of preprocessed signals of leads in a trial.

In step 5.4: obtaining a spatial feature vector for each trial by extracting a spatial feature of the preprocessed signal of each lead in the trial using a common spatial pattern.

The spatial feature may be used to characterize a spatial distribution of the preprocessed EEG signal across different leads. The processor may determine the spatial feature by computing a covariance matrix of the processed EEG signals for each trial.

The spatial feature vector may be a vector constructed from spatial features of the plurality of preprocessed EEG signals of leads in a trial. A spatial feature vector of 1*lead count (e.g., 1*30) may be obtained for each sample.

In some embodiments of the present disclosure, extracting the time-frequency feature set of the EEG signal of each lead based on the signal time-frequency plot of each lead, further obtaining the sample entropy feature vector, and further determining the spatial feature vector is conducive to obtaining dynamic changes of the EEG signal at different time and frequency and enhancing a nonlinear feature of the EEG signal.

In step 6: establishing an EEG classification model, and training and testing the established EEG classification model to recognize and classify a fuzzy semantic target in the natural reading environment.

The EEG classification model may be a model configured to recognize and classify the fuzzy semantic target in the natural reading environment.

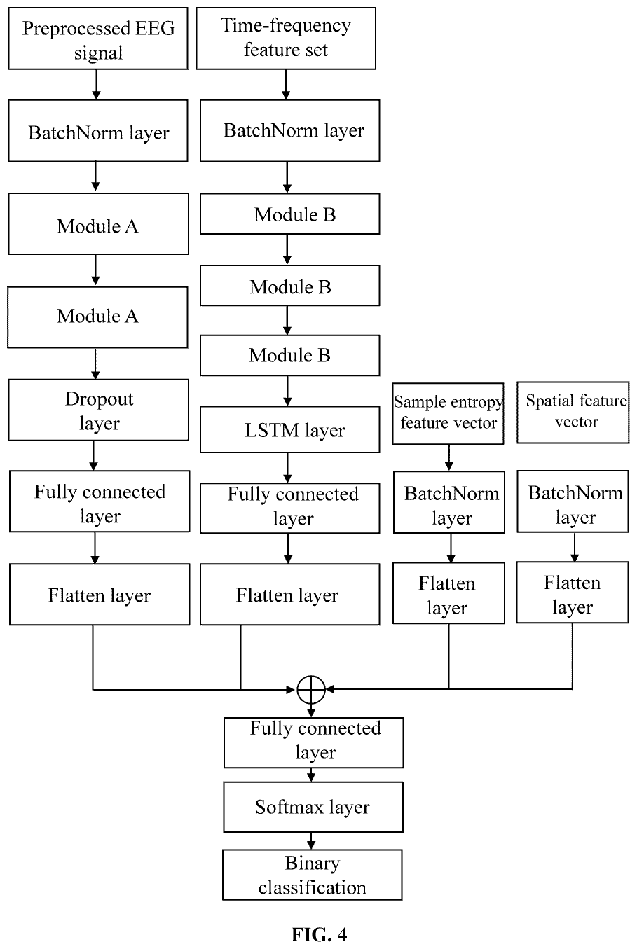

FIG. 4 is a schematic diagram illustrating an exemplary structure of an EEG classification model according to some embodiments of the present disclosure. As shown in FIG. 4, the EEG classification model may include an EEGNet module, a CNN-LSTM module, a temporal feature module, a spatial feature module, and an integration module.

The EEGNet module is configured to obtain a 1*30 feature vector using a preprocessed EEG signal obtained in step 4.4 as an input.

The CNN-LSTM module is configured to obtain a 1*30 feature vector by using a time-frequency feature set obtained in step 5.2 as an input and further extract a feature.

The temporal feature module is configured to obtain a normalized 1*30 feature vector by using a sample entropy feature vector calculated in step 5.3 as an input and normalize the sample entropy feature vector.

The spatial feature module is configured to obtain a normalized 1*30 spatial feature vector using a spatial feature vector obtained in step 5.4 as an input and normalize the spatial feature vector.

The integration module is configured to integrate the 1*30 feature vector obtained by the EEGNet module, the 1*30 feature vector obtained by the CNN-LSTM module, the normalized 1*30 feature vector obtained by the temporal feature module, and the normalized 1*30 spatial feature vector obtained by the spatial feature module into a whole integrated feature, input the whole integrated feature into a fully connected layer, learn and fuse the integrated feature using the fully connected layer, and finally obtain a binary classification output after processing by a Softmax activation function layer.

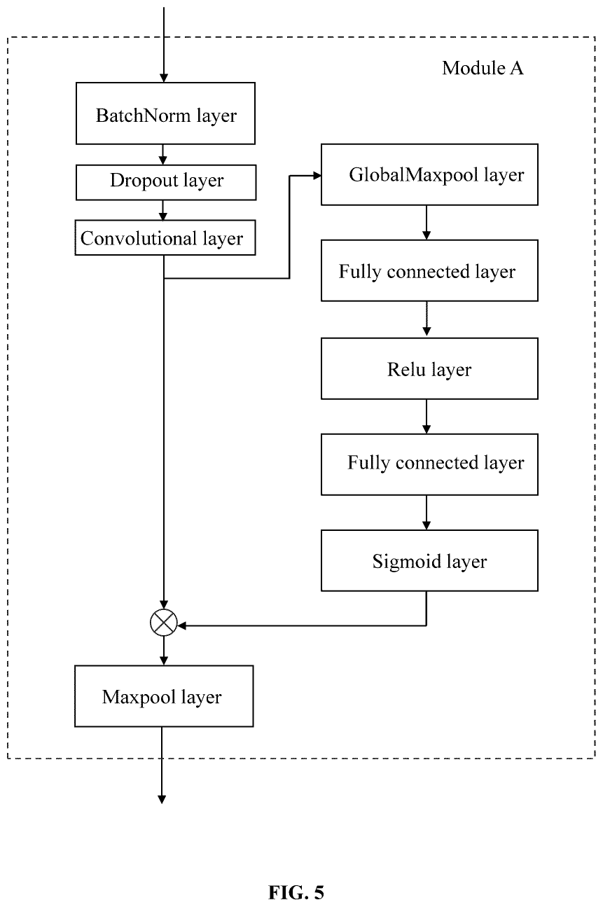

FIG. 5 is a schematic diagram illustrating an exemplary structure of a module A in an EEG classification model according to some embodiments of the present disclosure.

An EEGNet module may include a Batchnorm layer, two Modules A, a Dropout layer, a Fully connected layer, and a Flatten layer in turn. As shown in FIG. 5, each of the two Modules A may include a BatchNorm layer, a Dropout layer, a convolutional layer, a GlobalMaxpool layer, a Fully connected layer, a Relu layer, a fully Connected layer, a Sigmoid layer, and a Maxpool layer in turn.

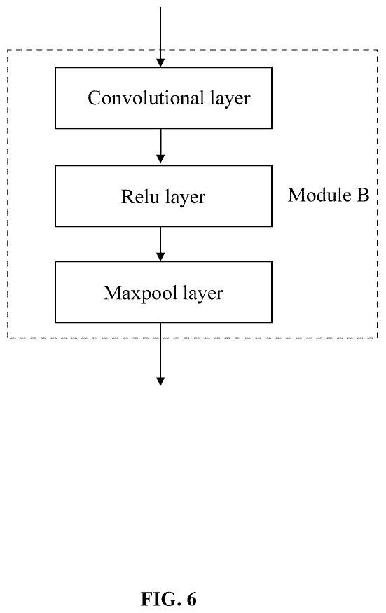

FIG. 6 is a schematic diagram illustrating an exemplary structure of a module B in an EEG classification model according to some embodiments of the present disclosure.

A CNN-LSTM module may include a BatchNorm layer, three Modules B, an LSTM layer, a Fully connected layer, and a Flatten layer. As shown in FIG. 6, each of the three Modules B may include a convolutional layer, a Relu layer, and a Maxpool layer in turn.

The integration module may include a Fully connected layer, a Softmax layer, and binary classification.

In some embodiments, the step 6 may include step 6.1 to step 6.3.

In step 6.1: randomly dividing EEG signal samples in an EEG database into a training set and a test set, using EEG data as an input and a corresponding label as a target output.

The training set may be a dataset configured to train internal parameters of a model. The test set may be a dataset configured to test a generalization ability of the model. After adjusting the internal parameters of the model using the training set, the test set may be used to determine whether the model is running properly and how well the model performs.

In some embodiments, the processor may randomly divide the EEG signal samples in the EEG database into the training set and the test set. A label corresponding to a sample indicates whether a target word appears in a stimulus material corresponding to an EEG signal sample.

In step 6.2: training the EEG classification model using the EEG data and the corresponding label of the training set to implement a binary classification of whether a target word appears.

A binary classification result may include the target word appears and the target word does not appear.

In step 6.3: applying the trained EEG classification model to the test set, and analyzing performance and robustness of the trained EEG classification model based on a classification result of the test set to explore an association between an EEG activity and a fuzzy semantic target recognition activity.

In some embodiments of the present disclosure, training the EEG classification model through the training set and the test set is conducive to improving the accuracy of the binary classification of the trained EEG classification model, thereby improving the generalization ability and robustness of the trained EEG classification model.

In some embodiments, the processor may also divide the training set or test set based on a count of rejections.

The processor may divide a plurality counts of rejections corresponding to a plurality of EEG experiments into a plurality of levels according to a gradient (e.g., a count of rejections within 10 times is classified as a level A, a count of rejections between 10 and 50 times is classified as a level B, etc.), and EEG signals corresponding to the plurality of levels are divided into a plurality of groups, and samples may be randomly extracted from EEG signals in the plurality of groups in accordance with a preset ratio into the test set and the training set. The preset ratio may be set by default by the processor or preset manually.

In some embodiments, the processor may evaluate a valid value of the EEG classification model based on an output of the EEG classification model.

The valid value may be a parameter that measures the performance of the EEG classification model. The valid value may be expressed as a numerical value, and the larger the valid value, the better the performance of the EEG classification model.

In some embodiments, the processor may determine a ratio of a count of concordant and a total count of labels, multiplied by an evaluation factor, as the valid value. The count of concordant may be a count of tests in which the output of the model is consistent with the label across a plurality of tests of the model.

In some embodiments, evaluation factors for different test sets in the step 6.3 are different, and the evaluation factor for the test set correlates to the count of rejections. The higher the count of rejections corresponding to EEG signal samples in a test set, the smaller the evaluation factor corresponding to the test set.

In some embodiments of the present disclosure, determining the evaluation factor based on the count of rejections enables accurate judging of the EEG classification model even when the sample quality is poor.

In some embodiments of the present disclosure, classifying EEG signals first and then extracting the EEG signals based on a discrimination threshold and the count of rejections is helpful to safeguard a consistent distribution of levels of count of rejections in the training set and the test set, which effectively improves the training quality of the EEG classification model.

In some embodiments of the present disclosure, by determining the fuzzy semantic target recognition paradigm, conducting the EEG experiment and acquiring the EEG signal, constructing the EEG database based on high-quality EEG signals, extracting the feature of the EEG database, and constructing the EEG classification model, the accuracy of the binary classification is enhanced. When a subject is cognitively processing a certain type of text, an EEG signal is recorded from the surface of the skull, and a cognitive process of the brain is parsed by studying the EEG signal, so as to provide experimental bases for cognitive disorders and other diseases.

In addition, certain features, structures, or characteristics of one or more embodiments of the present disclosure may be suitably combined.

Some embodiments use numbers to describe the number of components, attributes, and it should be understood that such numbers used in the description of the embodiments are modified in some examples by the modifiers “about”, “approximately”, or “substantially”. Unless otherwise noted, the terms “about”, “approximately”, or “substantially” indicate that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should consider the specified number of valid digits and employ general place-keeping. While the numerical domains and parameters used to confirm the breadth of their ranges in some embodiments of the present disclosure are approximations, in specific embodiments, such values are set to be as precise as possible within a feasible range.

For each patent, patent application, patent application disclosure, and other material cited in the present disclosure, such as articles, books, manuals, publications, documents, etc., the entire contents of which are hereby incorporated by reference herein. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions, and/or use of terms in the present disclosure shall prevail.

1. A target recognition method based on an electroencephalogram (EEG) signal in a natural reading environment, the target recognition method being executed by a processor, comprising:
step (1): determining a fuzzy semantic target recognition paradigm based on a stimulus material and an experimental Block, wherein the fuzzy semantic target recognition paradigm includes a total of 8 Blocks, each Block of the total of 8 Blocks includes a plurality of trials using a stimulus material corresponding to each Block, the plurality of trials included in each Block are run under a same condition in a preset order, an interval exists between each trial of the plurality of trials, the stimulus material in the step (1) is a text of 15 to 20 words in length and includes a target semantic material and a non-target semantic material, the target semantic material includes four categories including a name of people, means of transportation, an animal, and a fruit, the non-targeted semantic material is selected from a neutral news report, and in a single Block, a ratio of the target semantic material trial to the non-target semantic material trial is 3:7;
step (2): performing an EEG experiment to a subject according to the fuzzy semantic target recognition paradigm and controlling a wireless EEG acquisition device to acquire the EEG signal based on a sampling parameter, wherein the sampling parameter includes a sampling combination of the subject and an acquisition period;
step (3): constructing an EEG database by combining the acquired EEG signal and a corresponding label, wherein the corresponding label includes whether a target word appears in a stimulus material corresponding to the EEG signal, and the step (3) includes:
step (3.1): calculating a power spectrum of each of a plurality of lead signals;
step (3.2): labeling a lead signal with a value greater than twice a standard deviation of an average power spectral energy as a bad lead signal and supplementing remaining lead signals using neighborhood interpolation;
step (3.3): calculating, in a single trial, a median of a variance of lead signals and a median of a difference between each lead signal and an average of the lead signals;
step (3.4): labeling a trial in which either of the two medians is greater than twice a standard deviation and rejecting EEG data corresponding to the trial, wherein the step (3.4) includes:
obtaining a count of rejections of each subject in a respective EEG experiment;
in response to a subject whose count of rejections exceeds a count threshold, re-conducting the EEG experiment on the subject, and controlling the wireless EEG acquisition device to re-obtain EEG data based on a regenerated sampling parameter, wherein a regenerating process of the regenerated sampling parameter includes:
generating a plurality of candidate sampling parameters;
determining a rejection probability of each of the plurality of candidate sampling parameters using a parameter determination model, the parameter determination model being a CNN; and
determine the regenerated sampling parameter based on the rejection probability; and
step (3.5): corresponding retained EEG data with a label corresponding to the EEG data, and constructing the EEG database;
step (4): obtaining a preprocessed EEG signal by performing preprocessing on the EEG signal in the EEG database;
step (5): performing feature extraction on the preprocessed EEG signal; and
step (6): establishing an EEG classification model to recognize and classify a fuzzy semantic target in the natural reading environment of the subject and provide experimental bases for cognitive disorders, wherein the natural reading environment includes semantically ambiguous text information, the fuzzy semantic target is a binary classification result, and the binary classification result includes the target word that appears or the target word that does not appear;
in the step (6), the EEG classification model includes:
an EEGNet module, configured to obtain a 1*30 feature vector using the preprocessed EEG signal as an input, wherein the 1*30 feature vector includes a row vector containing 30 numerical elements;
a CNN-LSTM module, configured to obtain the 1*30 feature vector using a time-frequency feature set as an input and further extract a feature;
a temporal feature module, configured to obtain a normalized 1*30 feature vector using a sample entropy feature vector as an input and normalize the sample entropy feature vector;
a spatial feature module, configured to obtain a normalized 1*30 spatial feature vector using a spatial feature vector as an input and normalize the spatial feature vector; and
an integration module, configured to integrate the 1*30 feature vector obtained by the EEGNet module, the 1*30 feature vector obtained by the CNN-LSTM module, the normalized 1*30 feature vector obtained by the temporal feature module, and the normalized 1*30 spatial feature vector obtained by the spatial feature module into a whole integrated feature, input the whole integrated feature into a Fully connected layer; learn and fuse the integrated feature using the Fully connected layer, and finally obtain a binary classification output after processing by a Softmax activation function layer;
wherein the EEGNet module includes a Batchnorm layer, two Modules A, a Dropout layer, the Fully connected layer, and a Flatten layer connected in turn, each of the two Modules A includes the BatchNorm layer, the Dropout layer, a convolutional layer, a GlobalMaxpool layer, the Fully connected layer, a Relu layer, the Fully connected layer, a Sigmoid layer, and a Maxpool layer connected in turn,
the CNN-LSTM module includes the BatchNorm layer, three Modules B, an LSTM layer, the Fully connected layer, and the Flatten layer connected in turn; each of the three Modules B includes the convolutional layer, the Relu layer, and the Maxpool layer connected in turn,
the temporal feature module includes the Batchnorm layer and the Flatten layer connected in trun;
the spatial feature module includes the Batchnorm layer and the Flatten layer connected in trun;
the integration module includes the Fully connected layer, the Softmax layer, and binary classification connected in trun; and
the Flatten layer of the EEGNet module, the Flatten layer of the CNN-LSTM module, the Flatten layer of the temporal feature module, and the Flatten layer of the spatial feature module are connect to the Fully connected layer of the integration module.

2-3. (canceled)

4. The target recognition method of claim 1, wherein the step (2) includes:
step (2.1): wearing the wireless EEG acquisition device for the subject and informing the subject of a target semantic category;
step (2.2): randomly presenting a stimulus material in a single Block until all materials in the single Block have been traversed, and connecting a preceding material and a following material using a blank frame; and
step (2.3): recording the EEG signal and the corresponding label when presenting each stimulus material and the EEG signal and the corresponding label during a period of the blank frame following each stimulus material;
after a short break, repeating the step (2.2) and the step (2.3), presenting a stimulus material in a next Block until all Blocks have been traversed.

5. (canceled)

6. The target recognition method of claim 1, wherein in the step (4), the preprocessing includes filtering, downsampling, and denoising.

7. The target recognition method of claim 6, wherein the step 4 includes:
step (4.1): obtaining a re-referenced EEG signal by re-referencing the EEG data using an average of all lead signals as a reference datum;
step (4.2): obtaining a filtered EEG signal by removing, from the re-referenced EEG signal, noise below 0.5 Hz and above 80 Hz and power-line interference at 50 Hz using a band-pass filter and a notch filter;
step (4.3): obtaining a downsampled EEG signal by downsampling the filtered EEG signal according to a sampling theorem; and
step (4.4): decomposing the downsampled EEG signal into a plurality of independent components using independent component analysis (ICA), calculating a frequency feature of each component; and removing bioelectrical artifacts and residual noise.

8. The target recognition method of claim 7, wherein the step (5) includes:
step (5.1): obtaining a signal time-frequency plot of each lead by performing time-frequency analysis on the preprocessed EEG signal using continuous wavelet transform (CWT);
step (5.2): obtaining a time-frequency feature set of an EEG signal of each lead by extracting an image feature of the signal time-frequency plot using a convolutional neural network;
step (5.3): obtaining a sample entropy feature vector of each trial by calculating a sample entropy feature of a preprocessed signal of each lead in the trial; and
step (5.4): obtaining a spatial feature vector of each trial by extracting a spatial feature of the preprocessed signal of each lead in the trial using a common spatial pattern.

9. The target recognition method of claim 1, wherein the step (6) includes:
step (6.1): randomly dividing EEG signal samples in the EEG database into a training set and a test set, using the EEG data as an input of the EEG classification model and the corresponding label as a target output;
step (6.2): training the EEG classification model using the EEG data and the corresponding label of the training set to implement binary classification of whether the target word appears; and
step (6.3): applying the trained EEG classification model to the test set, and analyzing performance and robustness of the trained EEG classification model based on a classification result of the test set to explore an association between an EEG activity and a fuzzy semantic target recognition activity.

10. The target recognition method of claim 1, wherein
the step (2) further includes controlling the wireless EEG acquisition device to acquire the EEG signal of the subject in different acquisition time feature, and the step (3) further includes:
determining a quality of the EEG signal based on a user-type feature, a acquisition time feature, and the EEG signal by using a quality assessment model, the quality assessment mode being a machine learning model; and
determining whether to reject the EEG signal based on the quality of the EEG signal.

11. The target recognition method of claim 10, wherein an input of the quality assessment model includes the stimulus material corresponding to the EEG signal.

12. The target recognition method of claim 10, wherein the step (2) further includes:
controlling the wireless EEG acquisition device to acquire the EEG signal of the subject in different ambient light intensity features and material carrier features, and the input of the quality assessment model further includes the ambient light intensity features and the material carrier features.

13. The target recognition method of claim 9, wherein the step (6) further includes:
dividing the training set or the test set based on the count of rejections; and
determining a valid value of the EEG classification model based on an output of the EEG classification model.

14. The target recognition method of claim 13, wherein evaluation factors for different test sets are different, and the evaluation factors for the test sets correlate to the count of rejections.
